(19) United States
(12) Patent Application Publication
(10) Pub. No.: US 2016/0022568 A1
(43) Pub. Date: Jan. 28, 2016

(54) COMPOSITION FOR ORAL CARE

(71) Applicant: THERANOVIS GMBH & CO. KG, Bingen (DE)

(72) Inventor: Ashokkumar Patel, Minnetrista, MN (US)

(73) Assignee: THERANOVIS GMBH & CO. KG, Bingen (DE)

(21) Appl. No.: 14/777,230

(22) PCT Filed: Mar. 17, 2014
(86) PCT No.: PCT/DE2014/100091

§ 371 Date: Sep. 15, 2015

(30) Foreign Application Priority Data
Mar. 18, 2013 (DE) 10 2013 004 521.5

(51) Int. Cl. A61K 8/97; A61K 8/34; A61K 8/39; A61K 8/92; A61Q 11/00

(52) U.S. Cl. CPC A61K 8/97; A61K 8/922; A61Q 11/00; A61K 8/39; A61K 8/34

(57) ABSTRACT

A composition for oral care is proposed, wherein the composition is an aqueous composition and, besides water, comprises at least peppermint oil, rosemary oil and alcohol. In the context of the present invention, it has been found that the composition according to the invention exhibits a surprisingly good effect in the control of biofilms on dental surfaces. Harmful bacteria can no longer accumulate on the dental surfaces. The composition according to the invention can be used for oral and/or dental care and/or for oral prophylaxis, and can be used in particular for dissolving and/or removing extracellular polymeric substances (EPS) on dental surfaces, and/or plaque, and/or tartar, and/or for modifying the pellicle layer on dental surfaces.

### REFERENCE TO RELATED APPLICATIONS

This application is a U.S. national stage application of International Patent Application No. PCT/DE2014/100091, filed Mar. 17, 2014, and claims the benefit of priority of German Application No. 10 2013 004 521.5, filed Mar. 18, 2013, the entire disclosures of which are incorporated herein by reference.

### FIELD OF THE INVENTION

The present invention relates to a composition for oral care, wherein the composition is an aqueous composition. Further, the invention relates to uses of said composition. Finally, a method for dissolving and/or for removing extracellular polymeric substances (EPS) on tooth surfaces and/or dental plaque and/or tartar and a method for modification of the pellicle layer on tooth surfaces are specified, each by using the composition according to the invention.

### BACKGROUND OF THE INVENTION

The destructive effects of caries on tooth surfaces and teeth in general are commonly known. Thus, around 90% of adults suffer from caries. Further, diseases of the gums (gingiva), in particular inflammatory effects, are widespread. Gingivitis can rapidly lead to a periodontitis. A causal link between the presence of dental plaque (plaque) and in particular of tartar and different diseases of the gingiva is generally accepted.

Brushing the teeth, twice or several times a day, is generally considered to be a basis for a suitable prophylaxis in order to at least limit the undesirable formation of plaque on the teeth surfaces and a bacterial infestation of the oral cavity on the whole.

However, the regeneration of the so-called pellicle layer on the tooth surfaces can be detected within a few minutes after brushing the teeth. The terms “pellicle layer” and “pellicles” are used synonymously here and in the following text. It is a precipitate of proteins of the saliva. The pellicle itself is of little concern for dental health. However, other microorganisms of the saliva flora will be settling on the pellicle in the further course, resulting in an increase in the thickness of the biofilm developed.

In this case, harmful microorganisms can settle said biofilm. Here, a matrix of extra-cellular polymeric substances (EPS) may develop, which is capable of embedding most different types of bacteria species onto the tooth surface. The developing cell layer is called dental plaque (plaque) and considerably increases incidences of caries.

Tartar may develop by absorbing and embedding inorganic materials (minerals) from the saliva. Generally, said tartar can't be removed with a toothbrush. Tartar fosters the incidence of the above-mentioned diseases of the gingiva.

There are compositions known from the prior art, the use of which might result at a minimum in the cleansing of tooth surfaces and thus in the containment or removal of plaque.

There are oral and dental care and cleansing means known from DE 10 2008 033 105 A1 and DE 10 2008 039 681 A1, which are preferably configured in the form of known toothpastes. Said means include particles suspended in a fluid carrier, which can comprise synthetic materials, among others. However, today the market shows a growing demand for products with natural ingredients. Furthermore, the application of said cleansing products known from the prior art is limited to the brushing of teeth, which, however, is followed by the regeneration of a biofilm described above which occurs after the brushing of the teeth. Finally, it is not always possible to brush one's teeth everywhere and to do so discreetly.

DE 200 13 336 U1 describes an oil-based gel for oral and dental care, which is intended to be applied for the prophylaxis and/or treatment of inflammable diseases of the periodontium and/or the tooth neck. To that end, the gel includes a formulation comprising different vitamins. However, the formulation of undesirable films on teeth surfaces was demonstrated with respect to said formulation.

### SUMMARY OF THE INVENTION

Thus, the object underlying the present invention is to alleviate at least one of the above-mentioned disadvantages.

Further, it is the object of the invention to contribute to the dissolution and/or to the removal of pellicles and/or dental plaque (plaque) and/or tartar from tooth surfaces.

Further, it is the object of the invention to contribute to the antibacterial treatment of the oral cavity, in particular of tooth surfaces and/or of the gingiva.

Further, it is the object of the invention to provide a composition for the oral care which entirely consists of natural ingredients or which largely consists of as much natural ingredients as possible.

Finally, it is the object of the invention to contribute to a simple application of a composition for the oral care.

A composition for the oral care having the features of claim 1 contributes to achieving at least one of the above objects.

Advantageous embodiments and further developments are indicated in the dependent claims.

Further, a use of a composition according to the invention as well as a method according to various dependent claims contribute to achieving at least one of the above-mentioned objects.

### BRIEF DESCRIPTION OF THE DRAWINGS

FIGS. 1A, 1B, 2A, 2B, 3A, 3B, 4A, 4B, 5, 6A, 5B, and 7 are photomicrographs of specimens of bovine tooth enamel.

FIGS. 8A and 8B are photographs of extracted teeth.

### DETAILED DESCRIPTION OF PREFERRED EMBODIMENTS

In the scope of the present invention, it was found that the composition for oral care according to the invention exhibits a surprisingly good effect on the inhibition, reducing and dissolving (eliminating) the biofilms on tooth surfaces. Surprisingly, the composition is very suited for controlling the formation of such biofilms on tooth surfaces or even preventing them entirely. Here, on the one hand the formation of pellicles is modified and any extracellular polymeric structures which are present are dissolved on the other hand. The regeneration of the mentioned biofilms is impaired sustainably after use.

The application of the composition according to the invention on the tooth surface reduces and modifies the formation of pellicles such that the adherence of bacteria is sustainably reduced. Furthermore, the capability of the adherent bacteria to divide themselves and thus to colonize the tooth surface is significantly reduced or disrupted.

As a result, the formation of dental plaque (plaque) can be controlled earlier or even prevented. Harmful bacteria can no longer accumulate on the dental surfaces. Surprisingly, it was further found that that the composition for oral care according to the invention can dissolve or remove dental plaque a short period after regular application (e.g. approx. 2-4 weeks).

Surprisingly, even tartar can first be softened and finally be removed.

In addition, the composition according to the invention shows a remarkable antibacterial effect on oral care. Thus, the composition not only affects the already mentioned types of tooth deposits on tooth surfaces, but it is also capable of acting against caries and diseases of the gingiva.

The composition according to the invention can be used as a homogeneous (single-phase) spray or gel in the oral cavity. This facilitates the application. A toothbrush is not mandatory for the application of the composition, but can be used as support if desired. An application as a mouthwash, paste or the like is alternatively possible.

The composition according to the invention may consist entirely of natural ingredients, achieving a desirable transparency for the consumer. The consumer does not have to deal with synthetic ingredients that are not known to him and the possible inconveniences and/or undesirable effects thereof. The ingredients of the composition according to the invention themselves are uncritical and act in a mild and compatible manner within the oral cavity.

Furthermore, the composition according to the invention can be produced at comparatively favorable costs.

In an advantageous first further development of the inventive concept the content of peppermint oil is 0.2-2 weight percent (wt. %) of the composition according to the invention. As an alternative, a content of peppermint oil of 0.2-5 wt. %, particularly 1.2-2 wt. %, can be preferred.

According to another preferred embodiment, the content of rosemary oil is 0.2-2.5 wt. %, particularly preferred 0.5-1.2 wt. %, of the composition.

Alternatively, a content of rosemary oil of 0.2 to 0.5 wt. % may be preferred.

As an alternative or in addition to the above-mentioned embodiments, the content of alcohol may preferably be 15-35 wt. % of the composition.

In another advantageous illustrative embodiment, the composition comprises neem oil, particularly having a content of 0.05-10 wt. %, and particularly having a content of 0.1-0.2 wt. %.

In another specific and advantageous embodiment, the composition further comprises thyme oil, in particular having a content of 0.05-0.5 wt. %, and in particular having a content of 0.1-0.2 wt. %.

According to another advantageous embodiment, the composition alternatively or additionally comprises grapefruit seed extract, in particular having a content of 0.05-1.5 wt. %, and particularly having a content of 0.1-1.0 wt. %.

Further, a composition which comprises grape seed extract is preferred, particularly having a content of 0.05-1.5 wt. %, and particularly having a content of 0.1-1.0 wt. %.

According to another advantageous further development, the composition alternatively or additionally comprises clove oil, particularly having a content of 0.1-4.0 wt. %, and particularly having a content of 0.25-2.0 wt. %.

In another advantageous further development, the composition alternatively or additionally comprises polyglyceryl-10 decaoleate, in particular having a content of 5-15 wt. %, and particularly having a content of 10 wt. %.

In another advantageous further development, the composition alternatively or additionally comprises polyglyceryl-10 pentaoleate, in particular having a content of 5-15 wt. %, and particularly having a content of 10 wt. %.

In another advantageous further development, the composition alternatively or additionally comprises polyethylene glycol (PEG), in particular PEG 3350. The content of PEG can preferably be 5-20 wt. %, particularly preferred 15 wt. %.

In another advantageous embodiment, the composition alternatively or additionally comprises cyclomethicone, particularly having a content of 10-12.5 wt. %.

In another advantageous aspect of the invention, the composition alternatively or additionally comprises ceramide III, in particular having a content of 7.5-12.5 wt. %, and particularly having a content of 10 wt. %.

Further proven to be advantageous is a composition which alternatively or additionally comprises butyl stearate, particularly having a content of 5-30 wt. %, and particularly having a content of 10-25 wt. %.

In another advantageous further development, the composition alternatively or additionally comprises plankton extract, preferably having a content of 7.5-12.5 wt. %, particularly preferred having a content of 10 wt. %.

In another advantageous aspect of the invention, the composition alternatively or additionally comprises lecithin, in particular having a content of 7.5-12.5 wt. %, and particularly having a content of 10 wt. %.

Within the composition according to the invention, the alcohol may preferably comprise ethyl alcohol, particularly denatured ethyl alcohol.

Further, an embodiment is preferred where the water comprises deionized water.

The composition according to the invention proves to be particularly advantageous if it is configured as a formulation for spraying or as a spray, or as a mouth rinse.

In an alternative, a configuration as a lozenge, pastille, hard candy or chewing gum proves to be advantageous as well. Another special configuration concerns a dental adhesive strip. Such strips, equipped with a suitable active ingredient, are necessarily known to cause a lightening, respectively a bleaching of the teeth. In the scope of the present invention, the composition according to the invention should be brought into permanent contact to the tooth surfaces by means of such adhesive strips, if possible.

As an alternative, but also preferred, the composition may be a gel or formed gel-like, in particular wherein the composition comprises at least one gelling agent, preferably polyacrylic acid and/or xanthan.

Here, according to another specific further development, the composition may comprise polyacrylic acid as a gelling agent having a content of 0.2-1.0 wt. %, particularly 0.5 wt. %.

Further, according to a further non-limiting illustrative embodiment, the composition comprises a content of triethanolamine of 0.2 to 1.0 wt. %, particularly 0.5 wt. %. Triethanolamine, on the one hand, can serve as a gelling agent for a configuration as a gel; on the other hand, triethanolamine can be used to adjust the pH level and/or to adjust viscosity in the embodiments of the compositions as sprays, rinses or the like.

In the scope of other preferred optional configurations, the composition alternatively or additionally comprises oregano oil and/or tamanu oil and/or green tea extract.

In the scope of the invention, the use of the composition according to the invention is proposed for oral care and/or dental care and/or for oral prophylaxis.

Further, in the scope of the invention, use of the composition according to the invention is proposed for dissolving and/or for removing extracellular polymeric substances (EPS) on tooth surfaces, and/or dental plaque (plaque), and/or tartar. Furthermore, use of the composition for the modification of the pellicle layer on tooth surfaces is proposed. The applications mentioned can be carried out at the same time, where applicable.

Finally, a method according to the invention for dissolving and/or removing extracellular polymeric substances (EPS) on tooth surfaces and/or dental plaque (plaque), and/or tartar is indicated, wherein the method comprises the application of the composition according to the invention in the oral cavity and in particular on tooth surfaces.

Another method according to the invention relates to the modification of the pellicle layer on tooth surfaces, wherein the method comprises the application of the composition according to the invention in the oral cavity and in particular on tooth surfaces.

The methods mentioned can be performed simultaneously as well, where applicable.

The advantages of the applications according to the invention or the method according to the invention compared to known art in the respective categories can already be seen from the above explanations on the nature and advantages of the composition according to the invention, to which reference is made in full in order to avoid repetitions.

Furthermore, advantageous embodiments and further developments of the applications according to the invention or of the methods according to the invention also result from the above explanations of advantageous embodiments and further developments of the composition according to the invention. Here, features of different advantageous embodiments of the composition may be combined as well in the scope of advantageous further developments of the applications or the methods.

### EXAMPLES

Exemplary embodiments of the invention, which however do not limit the invention to the embodiments illustrated, are shown in the context of the following tables of exemplary compositions according to the invention. At the same time, said exemplary compositions—additionally or complementary to the subject matter of the dependent claims—indicate particularly preferred embodiments of the composition according to the invention.

All numerical values are to be understood as weight percentages (wt. %), based on the total weight of the composition.

The exemplary compositions indicated below are generally configured as a spray or as a solution, respectively, except compositions 7 and 8, which are configured as gels or gel-like compositions by adding a gelling agent.

| | Example 0 | Example 1B |
|---|---|---|
| Water (deionized) | 72.25 | 53 |
| Ethyl alcohol(denatured) | 25 | 35 |
| Peppermint oil | 1.2 | 5 |
| Rosemary oil | 1.2 | 5 |
| Grapefruit seed extract | 0.1 | — |
| Grape seed extract | 0.1 | — |
| Thyme oil | 0.05 | — |
| Neem oil | 0.1 | — |
| Clove oil | — | 2 |

| | Example 1 | Example 2 | Example 3 |
|---|---|---|---|
| Water (deionized) | 55.6 | 35.6 | 86.3 |
| Ethyl alcohol (denatured) | 35 | 60 | 12.5 |
| Peppermint oil | 5 | 5 | 0.2 |
| Rosemary oil | 2 | 2 | 0.2 |
| Grapefruit seed extract | 1 | 1 | 0.2 |
| Grape seed extract | 1 | 1 | 0.2 |
| Thyme oil | 0.2 | 0.2 | 0.2 |
| Neem oil | 0.2 | 0.2 | 0.2 |

| | Example 4B | Example 4 |
|---|---|---|
| Water (deionized) | 62.6 | 64.5 |
| Ethyl alcohol (denatured) | 35 | 35 |
| Peppermint oil | 1.2 | 0.1 |
| Rosemary oil | 1.2 | 0.1 |
| Grapefruit seed extract | | 0.05 |
| Grape seed extract | | 0.05 |
| Thyme oil | | 0.1 |
| Neem oil | | 0.1 |

| | Example 4C | Example 5 | Example 6 |
|---|---|---|---|
| Water (deionized) | 64.5 | 38.8 | 63.8 |
| Ethyl alcohol (denatured) | 35 | 60 | 35 |
| Peppermint oil | 0.1 | 1 | 1 |
| Rosemary oil | 0.1 | 0.1 | 0.1 |
| Grapefruit seed extract | 0.1 | 0.1 | 0.1 |
| Grape seed extract | 0.1 | — | — |
| Thyme oil | 0.05 | — | — |
| Neem oil | 0.05 | — | — |

| | Example 7 | Example 8 |
|---|---|---|
| Water (deionized) | 60.1 | 34.6 |
| Ethyl alcohol (denatured) | 35 | 60 |
| Peppermint oil | 1 | 5 |
| Rosemary oil | 0.5 | 2 |
| Grapefruit seed extract | 1 | 1 |
| Grape seed extract | 1 | 1 |
| Thyme oil | 0.2 | 0.2 |
| Neem oil | 0.2 | 0.2 |
| Polyacrylic acid | 0.5 | 0.5 |
| Triethanolamine (TEA) | 0.5 | 0.5 |

| | Example 11 | Example 12 | Example 13 |
|---|---|---|---|
| Water (deionized) | 62.6 | 62.6 | 50.1 |
| Ethyl alcohol (denatured) | 25 | 25 | 25 |
| Peppermint oil | 1.2 | 1.2 | 1.2 |
| Rosemary oil | 1.2 | 1.2 | 1.2 |
| Polyglyceryl-10 decaoleate | — | 10 | — |
| Polyglyceryl-10 pentaoleate | 10 | — | — |
| Cyclomethicone | — | — | 12.5 |

| | Example 14 | Example 15 | Example 16 |
|---|---|---|---|
| Water (deionized) | 57.6 | 62.6 | 62.6 |
| Ethyl alcohol (denatured) | 25 | 25 | 25 |
| Peppermint oil | 1.2 | 1.2 | 1.2 |
| Rosemary oil | 1.2 | 1.2 | 1.2 |
| Polyethylene glycol | 15 | — | — |
| (PEG)3350 | | | |
| Plankton extract | — | — | 10 |
| Cyclomethicone | — | 10 | — |

| | Example 19 | Example 20 |
|---|---|---|
| Water (deionized) | 47.6 | 59.75 |
| Ethyl alcohol (denatured) | 25 | 25 |
| Peppermint oil | 1.2 | 2.5 |
| Rosemary oil | 1.2 | 2.5 |
| Clove oil | — | 0.25 |
| Butyl stearate | 25 | 10 |

### Experiments

Test results, which were obtained by means of selected compositions from the previous section “Examples”, will be discussed below.

Here, reference is also made to the corresponding attached figures, which illustrate the obtained results compared to control images without previous use of a composition according to the invention.

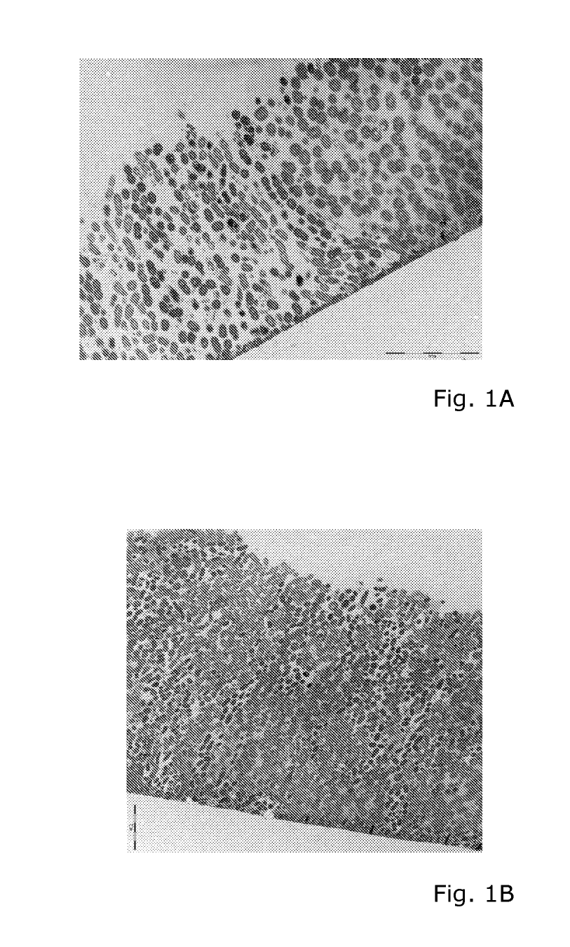

FIGS. 1-7 show electron-microscopically obtained top views or sectional views, respectively, of uniformly prepared specimens of bovine tooth enamel (tooth enamel specimens), which have been worn by test subjects (in vivo) in the oral cavity. The respective experimental conditions are explained by means of specific figures. FIGS. 1B, 2B, 3B, 4B and 6B in each case show control images under the same experimental conditions but without the use of a solution according to the invention for comparison purposes.

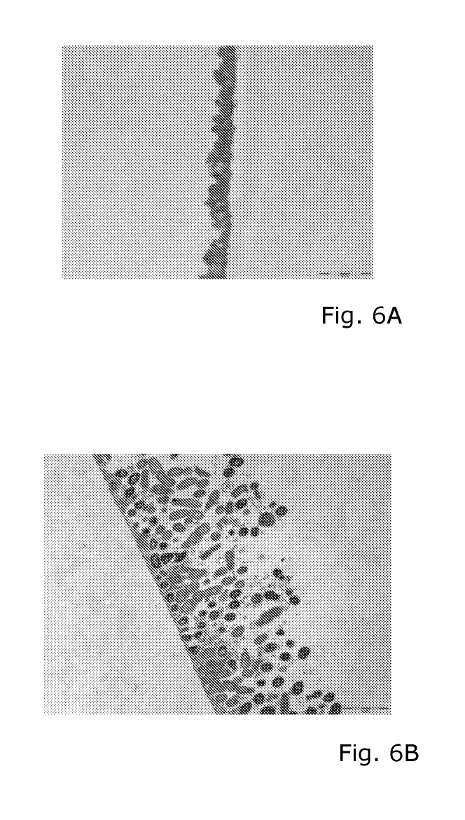

The cross-sectional images of the tooth enamel specimens according to FIGS. 1 and 6, illustrate that the enamel in each case was dissolved for the transmission electron microscopic analysis in the context of the preparation of specimens and is therefore no longer visible at the base of the biofilm.

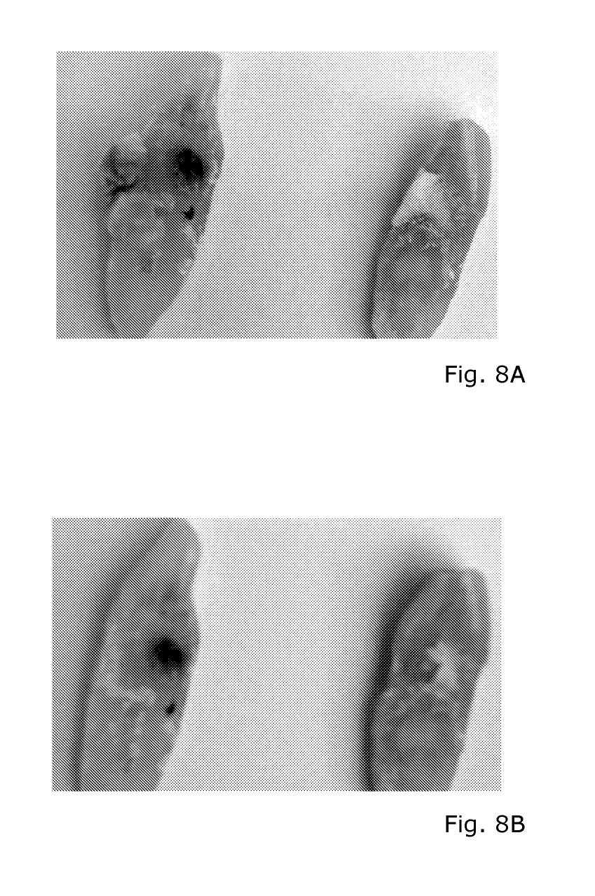

FIGS. 8A and 8B show photographs of two extracted human teeth originally having heavily developed tartar, prior to (FIG. 8A) and after (FIG. 8B) the use of a solution according to the invention.

Experiment 1: Example for the Dissolution of the Biofilm Matrix (the Extracellular Polymeric Substances, EPS):

Under oral cavity conditions, a dense bacterial biofilm forms on the surface of the tooth enamel within 48 hours, provided that any oral hygiene measures are not taken (see FIG. 1B, which shows a respective control image after 48 hours without the use of the composition according to the invention).

By means of a transmission electron microscope, mostly globular bacteria can be identified in the cross-section through the biofilm. The biofilm shows a dense, compact ultra structure, the cavities between the bacteria are filled up with inter-bacterial matrix.

The enamel was removed in the context of the sample preparation for the transmission-electron-microscopic analysis and is therefore no longer visible on the base of the biofilm.

If this 48-hour biofilm, according to FIG. 1B, is exposed outside the oral cavity in a solution according to the invention having the composition of example 4 (not: 4B or 4C) of the previous section entitled “Examples” for a duration of 1 hour, the outer bacterial layers of the biofilm are detached and the inter-bacterial matrix is removed. This results in a “loosened” ultra structure of the biofilm in the transmission-electron-microscopic image.

This result is illustrated by means of the respective picture according to FIG. 1A.

Experiment 2: Example for the Removal of the Biofilm Matrix:

After a 100-hour exposure in the oral cavity (without any oral hygiene measures) a dense film of adherent microorganisms forms on the surface of the tooth enamel surface or respectively on the surface of the tooth enamel specimen.

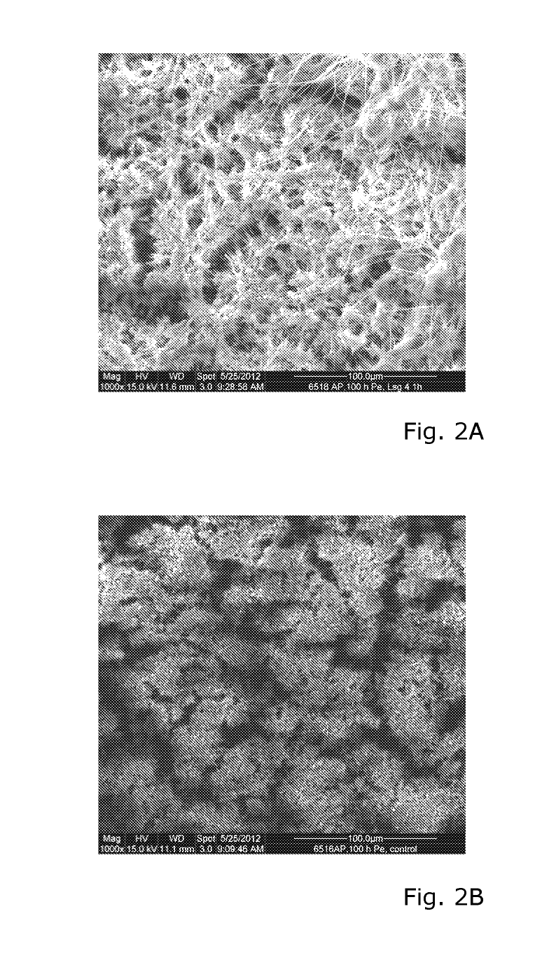

In a scanning electron microscope image or rather in the scanning electron microscope top view, this biofilm is a bacterial multilayer and a dense cover over the surface, traversed by cracks due to drying. Individual filament-shaped, rod-shaped and coccus-shaped bacteria can partially be identified on the surface of the biofilm. (See FIG. 2B, which shows a control image under to these conditions without the use of a composition according to the invention).

When exposed outside the oral cavity for 1 hour into a solution according to example 4, a loosened, broken-up, reticular surface structure results when examined in a scanning-electron-microscopic top view. The filament-shaped, rod-shaped and globular bacteria can clearly be detected, the interbacterial matrix is removed. This result is illustrated by the corresponding picture of the respective specimen according to FIG. 2A in a complementary manner.

Experiment 3: Example of Reduced Biofilm Regeneration:

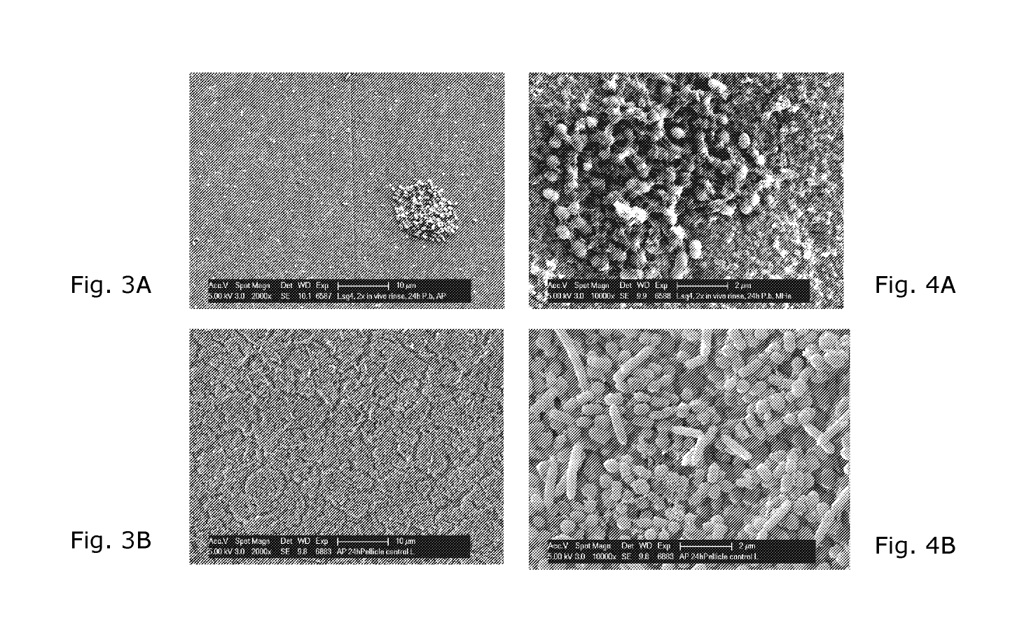

A dense biofilm (“bacterial lawn”) develops under oral cavity conditions on the surface of the tooth enamel within 24 hours without oral hygiene measures. In the scanning-electron-microscopic top view and higher magnification, globular and rod-shaped bacteria are visible which cover the tooth enamel surface as a multilayer biofilm and completely mask it. This is illustrated in the control images according to FIG. 3B and FIG. 4B. Compared to FIG. 3B, FIG. 4B shows a five-fold magnified image. Said size ratio also applies to the comparison of FIGS. 3A and 4A (see the following paragraph).

If the enamel specimen is rinsed with the solution according to example 4 twice in 12 hour-intervals for 30 seconds each time during the 24-hour exposure in the oral cavity, the biofilm regeneration is significantly reduced. Only individual islands of adherent bacteria are visible on the enamel surface in the electron-microscopic top view, but not a compact biofilm. The tooth enamel surface is covered by a granularly appearing protein layer (pellicle layer). Said pellicle contributes to the partial masking of the tooth enamel surface. This particularly advantageous result is additionally illustrated by the respective photographs of a correspondingly treated specimen according to FIGS. 3A and 4A.

Experiment 4: Example for a Massively Reduced Biofilm Regeneration and Pellicle Modification:

After rinsing two times for 30 seconds each time at 12-hour intervals with a solution having a composition of example 12 from the above section entitled “Examples”, the 24-hour biofilm formation is almost entirely prevented (inhibited) under oral cavity conditions. After 24 hours of intraoral exposure, the enamel surface is partially masked with a pellicle layer that appears in a mashed, granular manner in the electron-microscopic image after rinsing two times with the solution according to example 12. Only individual adherent bacteria (very scattered) can be detected on the pellicle.

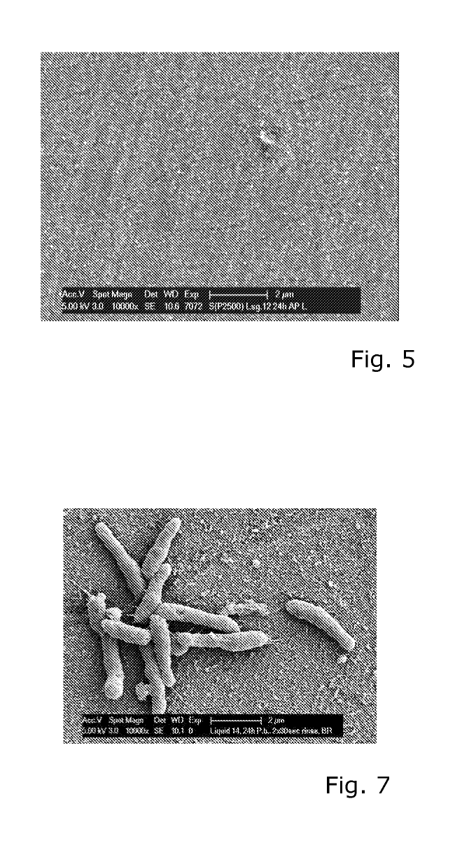

This result is illustrated by the respective picture of the correspondingly treated specimen according to FIG. 5 in a complementary manner.

Here, FIG. 4B again serves as a comparable control image without the use of a solution according to the invention.

Experiment 5: Example for Reduced Biofilm Reformation and Modified Pellicle Formation:

Also rinsing twice for 30 seconds each with a solution having the composition of example 14 from the above section entitled “Examples”, significantly reduces the 24-hour biofilm regeneration under oral cavity conditions compared to the control image (24-hour biofilm formation without intermediate tooth-brushing, see control image according to FIG. 4B). In the electron-microscopic view, only individual aggregates of adherent bacteria can be detected under the influence of rinsing twice with a solution according to example 14. A very dense and homogeneous pellicle layer covers the tooth enamel surface, which is proven by means of the picture of a correspondingly treated specimen surface according to FIG. 7. Here, the control image according to FIG. 4B again serves comparison purposes.

Further, the homogenous pellicle layer obtained by use of the solution according to the invention of example 14 which is discussed above appears as a granular surface coverage dense in electrons in the cross-section preparation when examined in a transmission-electron-microscopic view. This is illustrated by the respective picture of the cross-section preparation according to FIG. 6A.

The respective control image according to FIG. 6B shows the cross section through a control body, which has been obtained under the same conditions but without the use of a solution according to the invention. Compared to the homogenous pellicle layer according to FIG. 6A, FIG. 6B shows the formation of a multilayer bacterial biofilm.

Experiment 6: Example for the Dissolution and the Removal of tartar:

In the context of the present experiment, two extracted teeth having a severe coverage of tartar were treated with the solution according to example 14 (24 h-exposure). This leads to a “dissolution” of the bond between tartar and tooth structure; the adhesion of the tartar to the tooth surface is reduced. After being exposed to the solution according to example 14, the tartar can almost completely be brushed off/removed from the tooth surface by means of a toothbrush.

Usually, the tartar can only be removed from the tooth surface under the influence of considerable mechanical forces by means of a scaler or by use of ultrasonically driven tartar removal devices. FIGS. 8A and 8B show pictures of the extracted teeth, on one hand having severe tartar before the exposure in solution 14 (FIG. 8A) and on the other hand after carrying out the experiment explained above (FIG. 8B).

1. Composition for oral care, wherein the composition is an aqueous composition, and, besides water, comprises (in weight percentages):
Peppermint oil: 0.05-10%
Rosemary oil: 0.05-10%
Alcohol: 5-75%

2.-22. (canceled)

23. Composition according to claim 1, wherein the content of peppermint oil is 0.2-5 wt. %.

24. Composition according to claim 1, wherein the content of peppermint oil is 1.2-2 wt. %.

25. Composition according to claim 1, wherein the content of rosemary oil is 0.2-2.5 wt. %.

26. Composition according to claim 1, wherein the content of rosemary oil is 0.5-1.2 wt. %.

27. Composition according to claim 1, wherein the content of alcohol is 15-35 wt. %.

28. Composition according to claim 1, wherein the composition comprises neem oil in a concentration of 0.05-10 wt. %.

29. Composition according to claim 28, wherein the concentration of neem oil is 0.1-0.2 wt. %.

30. Composition according to claim 1, wherein the composition comprises thyme oil in a concentration of 0.05-0.5 wt. %.

31. Composition according to claim 30, wherein the content of thyme oil is 0.1-0.2 wt. %.

32. Composition according to claim 1, wherein the composition comprises grapefruit seed extract in a concentration of 0.05-1.5 wt. %.

33. Composition according to claim 32, wherein the content of grapefruit seed extract is 0.1-1.0 wt. %.

34. Composition according to claim 1, wherein the composition comprises grape seed extract in a concentration of 0.05-1.5 wt. %.

35. Composition according to claim 34, wherein the content of grape seed extract is 0.1-1.0 wt. %.

36. Composition according to claim 1, wherein the composition comprises clove oil in a concentration of 0.1-4.0 wt. %.

37. Composition according to claim 36, wherein the content of clove oil is 0.25-2.0 wt. %.

38. Composition according to claim 1, wherein the composition comprises polyglyceryl-10 decaoleate in a concentration of 5-15 wt. %.

39. Composition according to claim 1, wherein the composition comprises polyglyceryl-10 pentaoleate in a concentration of 5-15 wt. %.

40. Composition according to claim 1, wherein the composition comprises polyethylene glycol (PEG 3350) in a concentration of 5-20 wt %.

41. Composition according to claim 1, wherein the composition is configured as a preparation for spraying or as a spray or as a mouth rinsing solution.

42. Composition according to claim 1, wherein the composition is configured as a gel or gel-like composition, in particular wherein the composition comprises at least one gelling agent, preferably polyacrylic acid and/or xanthan.

43. Composition according to claim 1, wherein the composition comprises 1.2 to 2 wt. % of said peppermint oil, 0.5 to 1.2 wt. % of said rosemary oil, 15-35 wt % of said alcohol, 0.1 to 0.2 wt. % of neem oil, 0.1 to 0.2 wt % thyme oil, 0.1 to 1.0 wt. % grapefruit seed extract, and 0.1 to 1.0 wt. % grape seed extract.

44. Composition according to claim 42, wherein the composition comprises polyacrylic acid with a content of 0.2-1.0 wt %.

45. Composition according to claim 42, wherein the composition comprises triethanolamine with a content of 0.2-1.0 wt %.

46. Method for the dissolution and/or the removal of extracellular polymeric substances (EPS) on tooth surfaces,
and/or dental plaque (plaque)
and/or tartar,
wherein the method comprises the application of the composition according to claim 1 in the oral cavity, and particularly on tooth surfaces.

47. Method for the modification of the pellicle layer on tooth surfaces, wherein the method comprises the application of a composition according to claim 1 in the oral cavity and particularly on tooth surfaces.
